(19) United States
(12) Patent Application Publication
(10) Pub. No.: US 2012/0282320 A1
(43) Pub. Date: Nov. 8, 2012

(54) Hemostatic dressing

(75) Inventor: George H. Scherr, Park Forest, IL (US)

(73) Assignee: George H. Scherr Trust

(21) Appl. No.: 13/068,184

(22) Filed: May 5, 2011

(51) Int. Cl. A61L 15/00; A61K 47/36; A61K 38/39; B05D 7/00; A61K 31/34; A61K 31/195; A61P 17/02; B05D 5/00; C08B 37/06; A61K 31/716

(52) U.S. Cl. 424/445; 536/2; 514/777; 514/17.2; 514/58; 514/474; 514/561; 427/2.1

(57) ABSTRACT

The present invention relates to the preparation of a hemostatic dressing in the form of a powder that is particularly applicable for stemming severe bleeding and for incorporation of the powder into dressings that can stabilize the site of tissue injury, and simultaneously act as an antimicrobial agent.

### DESCRIPTION OF THE PRIOR ART

Methods to develop hemostatic dressings have been pursued for many years. Oxidized cellulose is a hemostatic dressing that is prepared from cellulosic products and had reasonably good hemostatic activity. Oxidized cellulose as a hemostatic agent has been described in The United States Pharmacopeia of America as early as 1970 (The United States Pharmacopeia of America, Eighteenth revision, Sep. 1, 1970).

Collagen, either extracted as porcine collagen or bovine collagen, has been used in hemostatic dressings. An absorbable collagen hemostat either in powder or sheet form has been prepared by Tainan Science Industrial Park. (Taiwan).

A collagen-alginate dressing is available from Ecom Merchandies.

Thrombin has also been used as a coagulation product (Wolberg, 2007); De Cristofaro et al., 2004).

Experiments performed as early as 1950 have shown that chitosan may be an effective cellular agglutinating agent. As a result of such early studies, it was shown that chitosan can agglutinate red blood cells. This agglutination results from the high negative charge on the membranes of the red blood cells and the net positive charge of the chitosan even in the presence of blood which has been treated with anticoagulants such as heparin. The use of chitin in agglutinating red blood cells can therefore also be used as a hemostatic agent.

Chitin is a polysaccharide that can be extracted from shellfish, clams, oysters, and other organisms. The deacetylation of chitin results in chitosan in which the —NHCO—CH₃ group of the chitin has been replaced with an acetamide group.

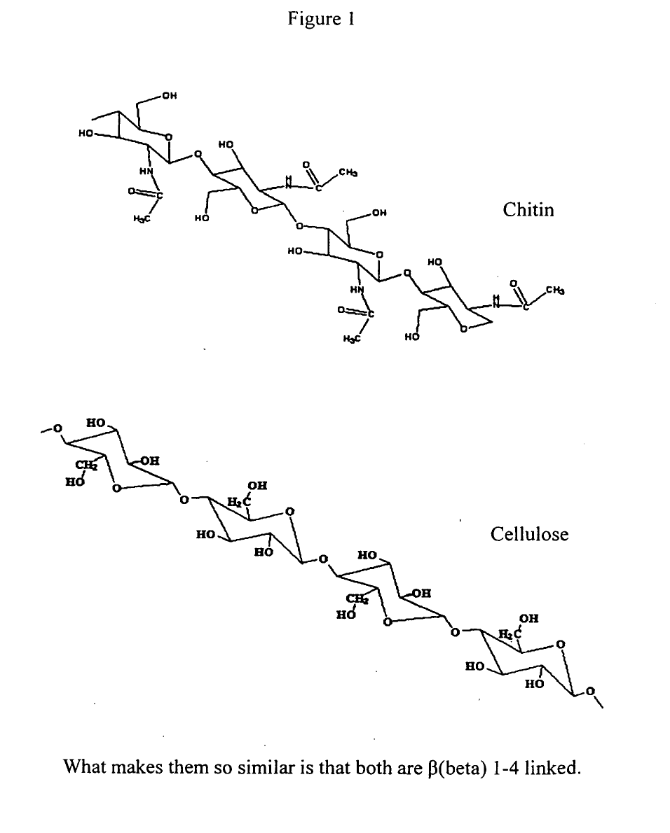

The molecular structure of chitin and cellulose are chemically similar and it would be expected that chitin and/or chitosan could be amenable for being introduced into a mixture in which the composition would contain other cellulosic molecules such as alginate (see FIG. 1).

A study performed by Pusateri et al. (2003) compared the hemostatic properties of a chitosan gauze with a standard cotton gauze sponge. Liver injuries were induced in swine and the dressings were applied 30 seconds later. In the chitosan-treated gauze group, the blood loss was reduced to approximately 264 ml, whereas in the group treated with the cotton gauze dressing, the blood loss was significantly greater; 2,879 ml. Consequently, the marked reduction of blood loss with the chitosan dressing resulted in a much higher survival rate for the swine thus treated in that seven of the eight swine in the chitosan-treated group survived, only two animals survived in the gauze-treated group.

Studies demonstrating the hemostatic capability of chitosan treated dressings warranted the comparison between the chitosan dressings and commercial collagen sponges and such a study was reported by Wang et al. (2006).

Wang et al. reported that, in comparative studies using a rabbit cervical vein wound, that the total amount of bleeding from the injured veins until hemostasis was achieved was essentially the same for both chitosan and collagen. Except for certain differences in the mechanical qualities of the experiment, the two sponges behaved similarly with regard to hemostasis. For example, the chitosan sponges were much more flexible and resistant to breakage than the collagen sponges and the chitosan sponge was degraded in situ much more slowly than the collagen sponges.

Rao and Sharma (1997) studied the safety and hemostatic potential of chitosan and reported that autoclaving was an applicable sterilizing method in that it caused the least decrease in tensile strength and affected the rate of hemolysis rather negligibly. In addition, they reported that sterilization of the chitosan with glutaraldehyde did in no way affect the maximum tensile strength of the chitosan. In vivo toxicity tests showed that the material was nontoxic and that sterilized films of the chitosan were free of pyrogen. The two workers reported that the hemostatic capability of chitosan was independent of the classical coagulation methods and appeared to be an interaction between the cell membrane, of the erythrocytes and chitosan. This observation was reported in greater detail in an attempt to explain how chitosan was acting as a hemostat. It is well known in the profession that the negative surface charge on red blood cells is principally due to the presence of neuraminic acid residues on the cell membranes. By removing the neuraminic acid with neuraminidase, it has been observed that chitosan would not then cause gelling of the red blood cells; it is therefore to be concluded that the gelling of the red blood cells is due to the interaction between the positively charged chitosan polymer with the neuraminic acid present on the surface of the red blood cells which provide a strong negative charge. It therefore, would necessarily follow that any alterations in the concentration of neuraminic acid would be reflected by an alteration in the avidity of the hemostatic agglutination of the red blood cells.

The hemostatic properties of chitosan have also been extensively reported by Malette et al. (1983).

Mi, F. L. et al., (2002) reported on a chitosan-containing dressing which was composed of two layers in which one of the layers contained silver sulfadiazine. The principal role of the chitosan was to act as a delivery system for silver sulfadiazine in which the layer containing the chitosan was to regulate the release of the silver sulfadiazine. The dressing was not designed and had little if any attributes that affected the coagulation of the bleeding wound.

Khan and Peh (2003) studied the effect of different chitosan films and a non-chitosan commercial dressing with regard to the rate of healing and the ease of film removal from punch biopsy wounds in rats. No studies were done nor were any preparations of chitosan made that were designed to assess the effectiveness of the chitosan to coagulate blood and/or stop bleeding.

The toxicity of chitosan when administered orally, as well as intravenously to experimental animals, has been shown to be extremely low. Thus, the LD₅₀ is in excess of 16 g/Kg in mice (See the 18^{th} Edition of Taber's Cyclopedic Medical Dictionary).

The use of chitosan in various pharmacological preparations has been described by Felt et al., 1998. Chitosan dressings prepared for humans have been in clinical use for the treatment of partial and full thickness dermal ulcers, leg ulcers, superficial wounds, abrasions, burns, and donor sites. Such chitosan dressings manufactured by the 3M Corporation under their trade name Tegasorb™ (Ilium, 1998; McRight, 1998).

The antimicrobial properties of chitosan have been reported against a number of microorganisms and probably are due to the properties of chitosan in acting as a coagulant of the microbial cells due to the strong positive charge of the chitosan molecule (Muzzarelli et al. 1988; Muzzarelli et al. 1990).

Soerens et al. (U.S. Pat. No. 6,967,261) reported the composition of a bandage that could be used for acute wounds or minor wounds in which a multiple layered dressing included a first layer for curbing the wound site, a second layer which was placed over the bottom surface of the first layer for absorbing exudates. The second layer included a poly (ethyleneoxide)-based compound as well as a chitosan-based compound. A third layer situated over the second layer, where the third layer was composed of a perforated film in which at least one antimicrobial agent was included in the bandage.

The company Biolife, L.L.C. manufactures a hemostatic powder. The QR (Quick Relief)® powder of Biolife is composed of a mixture of potassium iron oxyacid salt and a hydrophilic polymer. It therefore, attributes its action in coagulating blood to the hydrophilic nature of the polymer which will absorb the blood quickly due to the large surface area of the powder and form a protective clot over the wound. There is no indication in the literature of Biolife that it has any antimicrobial activity—see 510(k) K070520.

The directions for use of the Biolife hemostatic powder are to completely cover the wound and to apply pressure for 30 seconds which ultimately should form a protective scab.

The number of studies such as those of Malette et al. reported in his U.S. Pat. No. 4,394,373, showed that utilizing liquid or powdered chitosan, it was successful in forming a clot in a relatively short period of time. Thus, one ml of blood when placed in a test tube with descending aliquots of chitosan placed therein would clot in less than two minutes. A 0.8 ml of a chitosan solution placed in one ml of blood would clot in 3.5 minutes

The manufacturer of a chitosan dressing which was prepared by lyophilization by the HemCon company provided the directions for its use so that applying pressure of the dressing placed on a bleeding wound would require at least two minutes or even longer to achieve a cessation of bleeding. It is to be expected that the reports of Malette et al. in their U.S. Pat. No. 4,394,373 would provide completely different results than the results of using a HemCon dressing of lyophilized chitosan that may require two minutes of continuous pressure applied to the bleeding wound or even greater. Placing a few drops of citrated blood onto a HemCon lyophilized chitosan dressing did not result in the absorption of the blood by the chitosan surface of the HemCon dressing in an hour. This difference is doubtlessly due to the enormous difference in surface area available for the powdered chitosan of Malette et al. as described in his patent, and the utilization of a relatively hard surface of lyophilized chitosan with a surface area exposed to the blood that is considerably less than that available in a powdered product.

The use of the HemCon chitosan lyophilized dressing has resulted in an enormous outpouring of criticism by military medical personnel maintaining: “HemCon doesn't work. I have tried every one of these products many times, on many different kinds of wounds. For big-time bleeding—and that's what we're really worrying about here—HemCon doesn't work.” These statements were made by Navy Captain Peter Rhee, director of the Navy Trauma Training Center in Los Angeles. These statements were made public in the Nov. 21, 2005, issue of the Baltimore Sun newpaper.

The Z-Medica Corporation has developed a hemostatic agent called QuikClot. This product utilizes zeolite and contains silver ions in order to affect some antimicrobial activity. The literature for the QuikClot product indicates that its in vitro clotting time of whole blood “is not inferior in efficacy to the predicate device indicated in the USFDA 510(k) K070010. A claim that the clotting time is not inferior to predicate devices should be considered in light of the fact that the predicate devices stipulated by the company are previous QuikClot devices under their 510(k)'s K061767 and K021581. In addition, the antimicrobial activity indicated for in vitro testing results in “greater than a five log reduction of viable organisms within 60 minutes.”

The QuikClot package containing silver stipulates that the concentration of ionic silver is 26 mg/gm maximum. In addition, the 510(k) of the QuikClot product indicates that it takes less than 5 minutes to form a clot while whole blood without zeolite took longer than 9 minutes to form a clot.

The development of calcium alginate fibers by Courtaulds in Coventry, England, principally for the textile industry, prompted the investigation by researchers such as Scherr to prepare medical dressings from the calcium alginate fibers, especially in the light of their hemostatic activity and slight antimicrobial activity. (See U.S. Pat. No. 5,674,524, issued Oct. 7, 1997, entitled Alginate Fibrous Dressings and Method of Making; U.S. Pat. No. 5,718,916, issued Feb. 17, 1998, entitled Alginate Foam Products; U.S. Pat. No. 7,128,929, issued Oct. 31, 2006, entitled Alginate Foam Products.)

The increased interest in calcium alginate fiber dressings for the treatment of wounds further resulted in the incorporation of antimicrobial agents in such dressings. The resultant development by Scherr of a silver alginate moiety introduced antimicrobial activity for medical dressings that had a number of advantages over antibiotics. (See U.S. Pat. No. 6,696,077, issued Feb. 24, 2004, entitled Silver Alginate Foam Compositions; India Patent Number 221859, issued Jul. 8, 2008, entitled Alginate Foam Compositions; United Kingdom Patent Number GB 2,357,765, issued Apr. 21, 2004, entitled Alginate Foam Compositions; Russian Patent Number 2322266, issued Feb. 18, 2003, entitled Alginate Foam Compositions.)

Aqueous insoluble salts of alginate are readily achieved with salts of calcium, aluminum, zinc, copper, iron, and silver, in which case, the insoluble alginate salt is suspended in an aqueous gel. A detailed discussion of the properties of alginates and the preparation of their solid states has been published by McDowell in his book, Properties of Alginates.

Pectins, either alone or in conjunction with alginates have also been used in the preparation of products that are amenable to the manufacture of wound dressings. Pectins are polysaccharides that contain a 1,4-linked α-D-galactosyluronic acid residue. There are essentially three pectic polysaccharides that are isolated from primary cell walls and they can be characterized as:

1. Homogalacturonans

2. Substituted galacturonans

3. Rhamnogalacturonans

Pectins may be esterified with methanol and the pectins can be classified as high- or low-ester pectins. The low-ester pectins usually require calcium to form a gel. A detailed presentation of the chemistry of pectins has been published by the American Chemical Society and edited by Marshall L. Fishman and Joseph J. Jen (Chemistry and Function of Pectins, 1986).

Christian Bannert describes the preparation of a gel that can be utilized as a dressing into which gel disinfectants and medications can be added for treating the mucousa. These gels are obtained by using aqueous soluble alginates which can react with a calcium salt. Calcium salts can react with a pectin which has a low degree of esterification. (See U.S. Pat. No. 5,147,648, issued Sep. 15, 1992).

U.S. Pat. No. 5,688,923 by Gerrish issued Nov. 18, 1997, describes a process of making pectin fibers that can be utilized in wound dressings.

U.S. Pat. No. 3,639,575 issued February 1972 to Irving R. Schmolka is concerned with the treatment of burns utilizing water soluble silver salts and a matrix for those silver salts which are composed of aqueous gels of polyoxyethylene polyoxypropylene block copolymers.

All seven claims of the Schmolka patent require that the composition prepared is designed “ . . . to treat a burn wound.” A silver water soluble salt may be used which can be silver nitrate, silver sulfate, silver acetate, and silver lactate monohydrate; these silver salts must dissolve in water at a minimum concentration 0.1 percent.

The gels as prepared in the examples cited in U.S. Pat. No. 3,639,575 are transparent and form a clear gel at room temperature. When the gels are cooled, ostensibly below the temperature of room temperature, they become liquids.

The independent claim 1, upon which claims 2-6 of this patent are dependent, specifically sets forth that the matrix consists of a polyoxyethylene polyoxypropylene block copolymer having the formula HO(C₂H₄O)_{b}(C₃H₆O)ₐ(C₂H₄O)_{b}H wherein a is an integer such that the hydrophobe represented by (C₃H₆O) has a molecular weight of at least 2,250, and b is an integer such that the hydrophile portion represented by (C₂H₄O) constitutes from about 10 to 90 weight percent of the copolymer. U.S. Pat. No. 3,639,575 also sets forth that “A more detailed explanation of the preparation of these block copolymers may be found in U.S. Pat. No. 2,674,619 issued to Lester G. Lundsted on Apr. 6, 1954.

Studies examining the mechanism of action of silver in demonstrating antimicrobial activity led various workers to conclude that the presence of silver ions was more important than the amount of silver metal as colloidal silver or the time that the substrate containing microbes was exposed to the silver. Silver was prepared in the form of a spongy metallic form or by coating various products which contain large surfaces such as sand by K. Süpfle and R. Werner (1951 Microdeosimetric investigation of the oligodynamic effect of silver. Mikrochemie ver. Mikrochim. Acta., 36/37, 866-881). These workers showed that E. coli placed in flasks having counts of 18,000 E. coli per ml of water, when such counts were exposed to flasks containing sand coated with silver at a concentration of 10 percent silver of the amount of sand, would result in a sterile environment in four hours. Even counts as high as 120,000 E. coli per ml of solution, resulted in sterility in 24 hours. The relatively low count of E. Coli of 1.8×10³ and the very high concentration of silver coated onto the sand of 10 percent would readily explain a sterile environment in four hours. However, in vivo experiments reports in 510(k) reports that are available have never utilized a silver concentration as high as 10 percent which could be toxic to a patient and result in argyria.

Azary Technologies LLC has produced a silver-containing fabric in which a nylon-containing textile fiber which contains silver which has been plated onto the nylon. The Azary company in submitting data pertinent to their United States Food and Drug Administration 510(k) device, K040518, has indicated that the silver contained on the nylon fiber is pure elementary silver at a purity of 99.9 percent. These textiles coated with elemental silver have been approved for coating nylon which can be used in a medical dressing or in certain wearing apparel such as socks. Here too, it is expected that the elemental silver will have to be converted to ionic silver by some mechanism in order to ensure antimicrobial activity.

The biocidal composition including sodium polypectate described in James W. Van Leuven's U.S. Pat. No. 4,184,974 is used in the range of 100 to 400 parts per million, also includes in claim 1, a silver ion in the range from about 13 to 250 parts per million, and glycerin in the range of about 4 to 10 percent by weight; in addition, the pH of the water soluble base should be in the range of 7.2 to 7.8.

The inventor submits that the sodium polypectate may be prepared by treating pectin with sodium carbonate in order to solubilize the pectins. The inventor therefore characterizes pectin as a water insoluble compound which is inconsistent with data submitted by innumerable documents that describe the utilization of pectins. The American Chemical Society monograph, Chemistry and Function of Pectins, unequivocally states that “Pectins are soluble in pure water.” The only caveat the ACS monograph submits is the case where “ . . . they (pectins) are insoluble in aqueous solutions in which they would gel at the same temperature if dissolved at a higher temperature.” However, that characterization for pectins when they become insoluble, is totally irrelevant to the specification and claims of U.S. Pat. No. 4,184,974. The inventor of this patent also submits that the preparation of sodium polypectate in order to solubilized the pectin with sodium carbonate also serves to result in the polypectate chelating readily with the alkaline earth cations such as calcium and magnesium. The reaction of calcium with pectin is well known and has been described in innumerable documents. The American Chemical Society's Symposium on pectins cited above also characterizes on page 8 that pectin will gel in the presence of divalent cations and increasing the concentration of divalent cations, such as calcium, increases the gelling temperature and gel strength.

In the patent by Gerrish, et al. (U.S. Pat. No. 5,688,923 issued Nov. 18, 1997), the inventors show that pectin can readily be dissolved in water as is indicated for example, in their claim 29. Their claim 35 indicates pectins will react with polyvalent cations wherein the cation which may consist of calcium, copper, barium, magnesium, zinc, and iron and will precipitate as an insoluble fiber of a pectate. The inventors have made no claim nor conducted any experiments as described in their U.S. Pat. No. 5,688,923 concerning the ability of silver ions to precipitate pectins. Also, they claim in their claim 6, that their claim 1 indicates they have non-pectin polysaccharides added to it such as hyaluronic acid, carrageenan, alginic acid, or sodium alginate. Alginic acid is insoluble in water, as has been well described in the chemical literature, but sodium alginate when added to the pectin where a fiber is to be made will certainly enhance the strength of the fiber.

Consequently, it is not clear and inconsistent with known chemistry why U.S. Pat. No. 4,184,974 of Van Leuven finds it necessary to solubilized pectins, which are known to be soluble in water, and that the preparation of a sodium polypectate is deemed necessary in order for the pectin to chelate with alkaline earth cations such as calcium when pectins readily chelate with calcium and become insoluble as has been well described in the literature.

The seventh edition of The Merck Index indicates that pectin is “completely soluble in 20 parts of water forming a viscous solution containing negatively charged hydrated particles.” For use as a medical product, pectin per se, as a powder was recommended in The Merck Index for hemostatic effect and as a paster for treatment of decubitus ulcers.

The MSDS sheet of Spectrum Laboratory Products, Inc., Gardena, Calif., 90248 dated Sep. 13, 2006 essentially paraphrases the data for pectin that The Merck Index cited above as being soluble in 20 parts of water and dissolves more readily in water if it is first moistened with alcohol or glycerol. The synonyms for pectin manufactured by Spectrum Laboratory Products, Inc. indicates that it may be also cited as Methoxypectin, Methyl pectin, Methyl pectinate, Pectinate, Pectrinic acid, Pectins, and Colyer Pectin. Chapter 5, in the book by A. Nussinovitch provides an excellent review of the chemistry of pectins, their preparation, and their reactions.

A singular aspect of the chemical activity of silver and silver salts has to do with the ease with which silver ions combine with proteins, oxygen, and various halogens to result in insoluble material. Thus for example, oxides of silver (as Ag₂O or AgO) are considered insoluble in water as is silver chloride. L. Goodman and A. Gilman (The Pharmacological Basis of Therapeutics, 3^{rd} Ed. New York, The Macmillan Company 1965) reported that the toxic effects of silver compounds on microorganisms is probably due to the silver ions which precipitate the protein of bacterial protoplasm. It is very well known that soluble silver salts such as silver nitrate will quickly precipitate protein and oxidize to a dark brown or black precipitate. The silver protein complex so formed contributes to a sustained antimicrobial action by slowly liberating small amounts of silver ions. It therefore would be necessary for the silver metal, in the report by Illingworth, et al. for the device in repairing a diseased or damaged heart valve, to be ionized to the silver ionic state.

Neither silver ions nor silver colloids should be released into a wound in a relatively large amount in a short period of time; otherwise the patient will turn blue as a result of silver poisoning (argyria) (Trop et al., 2006).

### SUMMARY OF THE INVENTION

The invention described herein relates to the composition of a hemostatic powder that can be utilized as a wound dressing to stem bleeding and which also contains a silver pectate moiety for the inhibition of microorganisms. The pectin utilized should also be amenable to react with a soluble calcium salt and produce an insoluble calcium pectate in which the calcium aqueous soluble salt may be calcium chloride or calcium gluconate. The purpose of including calcium pectate within the preparation is to serve as a chemical impedance to the release of silver ions as the silver pectate moiety disassociates. By adjusting the amount of the calcium pectate that is contained within the preparation one can achieve a well-controlled release of the silver ions at a rate that would be commensurate with the use of the silver ions as an antimicrobial agent and also in a concentration low enough to avoid too high a concentration of silver ions being achieved in a relatively short period of time which could possibly result in argyria.

The use of a hemostatic powder as proposed in our patent would be particularly significant to an injury that results in severe bleeding, because this hemostatic powder prepared at approximately a 100 mesh size of particle would present a very large surface area to the blood. This would reduce the clotting time significantly as opposed to a lyophilized dressing which has a surface area through which the blood must be first permitted to diffuse to enter the dressing itself before bleeding can be reduced by the charges initially present only on the surface of the chitosan.

The dressing of the instant patent also has the advantage in that the coagulation of blood basically occurs due to the high hydrophilic nature of the polymer incorporated in the powder. Consequently, the hydrophilic powder is essentially designed to coagulate the liquid part of the blood which necessarily would trap the red blood cells. The role of the HemCon dressing or any dressing that utilizes the charge of the chitosan molecule neutralizing the charge on the red blood may be impaired by two issues. The charge on the red blood cells may be reacting with a charge on the chitosan molecule thus causing coagulation of the red blood cell, but this would play no role whatsoever in coagulating the fluid part of the blood in which the red blood cells are contained.

The neuraminic acid on the surface of the red blood cells is responsible for the charge on the red blood cells which ostensibly reacts with the positive charge on the chitosan. However, the negative charge contained by the neuraminic acid may vary and be influenced by charges on other ingredients of the composition and/or the charges of the neuraminic acid may be modified by pathological conditions in the red blood cells so rendering the red blood cells less amenable to reacting with the positive charge on the chitosan.

An ancillary attribute of the patent described here also makes feasible the incorporation of the hemostatic powder being included in the composition for a dressing which would also include the hydrophilic powder in a highly foamed matrix. Such a composition would readily lend itself to the cessation of bleeding if it is not too severe, since the foamed matrix would permit a more ready absorption of blood from a wound than a lyophilized hard surface of chitosan which does not have the porosity of a soft foamed product.

An additional attribute of the composition of the patent described herein has to do with the fact that any wound that results in bleeding and certainly one that results in severe bleeding would necessarily be amenable to infection from organisms that are present on the skin or in and on those elements that caused the wound. The addition of a silver ion-containing molecule which has been shown for a number of years to have excellent antimicrobial activity would be an asset to avoid the risk of disseminated infection from the site of any lesion. Further, even if the silver ions succeed in resulting in bacteriocidal activity in large numbers of bacteria in and on the wound, it has been shown by Silvetti 1981; Silvetti 1987; and Silvett±1993 that an excellent chemotactic agent which will result in leucocytic attraction to the site of an injury is the maltodextrin and such an ingredient has been contained in the basic hemostatic powder and the hemostatic dressing described herein.

The hemostatic dressings described herein do not rely on the neuraminic acid concentration on the red blood cell or the negative charge on the red blood cells to be coagulated when and if a positive charge from a molecule of chitosan can be brought into the blood and react with the red blood cells. The QuikClot device of the Z-Medica Corporation described herein claims that antimicrobial activity when tested in vitro will result in “greater than a 5 log reduction of viable organisms within sixty minutes.” Depending upon how the in vitro tests have been performed, in vitro determinations of antimicrobial activity frequently have little relationship to the actual hemostatic activity of a product when placed on an open wound. This obviously would have to do with the highly nutritive environment for microbial multiplication in a wound at body temperature and in which blood tissues would be available for the multiplication of microorganisms. Consequently, the testing of antimicrobial activity should be performed in a setting which either mimics or depends on the determination of such antimicrobial activity in an actual wound by inhibiting any microorganisms present therein. Further, a sixty-minute time element to achieve a 5 log reduction of viable organisms would be too long a time that would still risk a microbial turnover, especially with microorganisms that have a relatively low generation time and the potential risk of a systemic infection resulting there from.

Consequently, the determinations by the agents proposed herein show hemostatic activity to be far more favorable in reducing the time element of hemostasis and the number of organisms reduced in far less than sixty minutes.

One of the advantages discovered in preparation of the pectin composition the described herein utilizes the addition of sodium tetraborate to the pectin composition when a fixed dressing is prepared since the sodium tetraborate would enhance the elasticity of the dressing so prepared.

Another advantage of the invention described herein relates to the preparation of pectin compositions in which the aqueous portion of the composition can be removed by air-drying or regulated heat-drying without the necessity of utilizing an expensive freeze drying apparatus for their preparation.

Another salient advantage of the invention described herein concerns the feasibility of adding ingredients to the pectin composition which ingredients may contain properties such as being particulate, having high viscosity, or having or resulting in a rheology which makes it undesirable or unfeasible for such compositions to be forced through a fine spinneret to produce the pectin fibers as currently practiced in U.S. Pat. No. 5,688,923.

The antimicrobial moiety utilized for this patent is silver pectate. The silver ions will gradually be released from the silver pectate molecule from a dressing that has been prepared in which the silver pectate is contained in a composition in a matrix. The silver ions have been shown in innumerable reports of dressings that are made with silver ions to have broad antimicrobial activity. In addition, there has not appeared any definitive evidence that microbial resistance to silver ions by microorganisms has taken place. Consequently, all of the silver in the powdered or formed dressings described herein are in the ionic state.

The inclusion within the composition containing a silver pectate also includes calcium pectate which acts as an impediment to the migration of the silver ions in a treated wound. As such, the release of the silver ions can be regulated to release a relatively fixed amount of silver ions over a relatively short or long period of time.

Having set forth the tenets of the invention contained herein, the following non-limiting examples illustrate various compositions that are inherent in my invention:

### DESCRIPTION OF THE PREFERRED EMBODIMENTS

### Example I

The following ingredients were added in the order provided:

7 gm of silver pectate

300 ml of deionized or distilled water

1.5 gm of calcium gluconate

1 gm of sodium tetraborate (Borax)

1 gm of carboxymethylcellulose—high density

2 gm of a hydrophilic polymer 1 gm of maltodextrin

The pectin utilized in the preparation of the silver pectate has a degree of esterification (DE) of less than 30 percent and a degree of amidation (DA) of less than 50 percent.

The entire composition was mixed to ensure homogeneity and placed in a ceramic dish. The ceramic dish containing the entire composition was placed in an oven set at 85-90° C. and dried for 18 hours, resulting in a dried brittle solid. The entire dried contents were ground in a mill to a fine powder and the powder placed onto a 100 mesh screen. Any of the powder that passed through the 100 mesh screen was then collected and tested.

### Example II

One ml (1 ml) of normal citrated sheep blood was placed into each of 3 test tubes. Add 0.5 grams of the 100 mesh powder prepared in Example I into the first tube; clotting of the blood results in 8.5 seconds.

Placing 1 gm of the 100 mesh powder prepared in Example I into the second test tube resulted in clotting of the citrated blood in 6.0 seconds.

Placing 1.5 gm of the 100 mesh powder prepared in Example I into the third test tube resulted in clotting of the citrated blood in 4.0 seconds.

### Example III

Seven grams (7 gm) gm of the 100 mesh powder prepared as in Example I was added to 50 milliliters (ml) of deionized or distilled water.

| | | |
|---|---|---|
| Glycerin was then added in the amount of | 10 | ml |
| Tween 80 was added in the amount of | 3 | ml |
| Calcium gluconate was added in the amount of | 2 | gm |
| Na lauryl SO₄ | 1 | gm |
| Propylene glycol | 3 | ml |
| Maltodextrin | 1 | gm |
| Carboxymethylcellulose -low viscosity | 5 | gm |
| Hydrophilic polymer | 2.5 | gm |

The above ingredients were thoroughly mixed to ensure a foamed homogeneity and layered onto a fibrous cloth or alternatively layered into a thin ceramic dish. The dish was incubated at 40° C. for twelve hours at which time it was removed and the following tests were performed:

A drop of citrated sheep blood was dropped onto the surface of the dried composition and the time measured for its complete absorption into the dressing. With the composition as shown in this Example III, the entire drop of blood was completely absorbed in six seconds.

Preparing the same composition as in Example III, except adding 4.0 grams of hydrophilic polymer, all other ingredients remaining the same, a drop of citrated sheep blood was absorbed in 4.5 seconds.

The above descriptions and examples illustrate particular constructions including the preferred embodiments of the solutions. However, the invention is not limited to the precise constructions describer herein, but, rather, all modifications and improvements thereof encompassed within the scope of the invention.

Many of the examples described herein utilize the surface active agents such as those characterized as Tween 80 or sodium lauryl SO₄. These surface-active agents are utilized primarily to effect a homogenous dispersion between the non-aqueous soluble components with the aqueous soluble components in order to ensure homogeneity.

These surface active agents are also utilized in order to improve the wetting of a medical dressing or bandage in the event that a wound may be exudating, and the enhanced wicking in such a bandage or medical dressing serves to quickly absorb any blood or serum from a wound site into the dressing. Other surface active agents, such as a member of the group of Tweens: (Tween 20, polyoxyethylene sorbitan monolaurate; Tween 40, polyoxyethylene sorbitan monopalmitate; or Tween 85, polyoxyethylene sorbitan trioleate may be incorporated into the pectin composition without deviating from the novelty of the invention described herein.

The use of glycerin or propylene glycol provide a soft texture of the gel composition and are well known in the profession; their attributes of which have been recorded in the past such as in U.S. Pat. No. 6,696,077 B2. The concentrations of glycerin or propylene glycol are provided here in order to effect the texture and softness of the gel dressing as prepared as described herein, but it is clear that other concentrations of the glycerin or propylene glycol may be provided without in any way deviating from the hemostatic activity for which the dressing has been principally compounded. It is well known in the profession that various glycols will act as plasticizers and may be used to improve the flexibility of gels or fibers. The plasticizer that we have principally used in the examples described herein has been glycerin because of its low cost, low toxicity, and ready availability. It is clear, however, that other plasticizers may be utilized such as ethylene glycol without deviating from the novelty of the invention described herein.

In the examples cited herein, calcium gluconate has been utilized to provide the calcium ions which precipitate the aqueous insoluble calcium pectate which insolubilization may also serve to entrap and/or restrain the release and migration of the silver ions from the silver pectate matrix as described herein. It is clear, as has been mentioned, that other salts may be utilized to precipitate the pectins such as those of aluminum, zinc, copper or chromium. Such insoluble pectates may be readily utilized to precipitate the various mixtures described in the examples provided herein without deviating from the essential merits of this invention. However, if the pectate compositions are to be utilized in or on biological tissues, the particular salt utilized to precipitate the pectin should be dictated by any restraints of toxicity or other untoward reactions that might result from their use for the preparation of hemostatic dressings.

In the examples cited, the pectin had a degree of esterification of less than 30 percent in order to achieve a relatively high degree of reactivity with calcium and other ions. It is clear however, that pectins other than those having a degree of esterification of less than 30 percent may be utilized without deviating from the essential tenets of the invention.

In the examples cited the silver pectate composition of Example I was dried in an oven set at 85-90° C. Temperatures other than 85-90° C. may be utilized to dry the subject composition without deviating from the essential tenets of the invention.

In the example cited, the silver pectate dried contents were ground in a mill to a fine powder which was then passed through a 100 mesh screen. The particle size of the powder may be placed through a mesh screen achieving larger or smaller particle sizes without deviating from the essential tenets of the invention. It is sufficient to indicate that the smaller the mesh size utilized would therefore provide a powder having a much higher surface area that is expected to improve the speed of the hemostatic activity.

### REFERENCES

Azary Technologies LLC 510(k) K040518
Biolife, L.L.C., for QR—510(k) K070520
De Cristofaro R, De Candia E (2004). “Thrombin domains: structure, function and interaction with platelet receptors”. J. Thromb. Trombolysis 15 (3): 151-63.
Ecom Merchandies, 73-26 Yellowstone Boulevard, Suite #26, Forest Hills, N.Y.
Felt, O. Buri, P., & Gurny, R. (1998) Chitosan: A unique polysaccharide for drug delivery. Drug Dev. Ind. Pharm. 24(11) 97-993.
Fishman, Marshall L. and Joseph J. Jen—Editors—Chemistry and Function of Pectins, 1986.
Goodman, L. and A. Gilman (1965 The Pharmacological Basis of Therapeutics, 3^{rd} Ed. New York, The Macmillan Company)
Illingworth, et al. (J. Heart Valve Dis. 1998 September; 7(5): 524-30)
Ilium, L. (1998) Chitosan and its use as a pharmaceutical excipient, Pharm, Res., 15(9), 1326-1331
Khan, Tanveer Ahmad and Peh, Kok Khiang—“A preliminary investigation of chitosan film as dressing for punch biopsy wounds in rats” J Pharm Pharmaceut Sci 6(1):20-26, 2003
Malette, W.; Quigley, H.; Gaines, R.; Johnson, N.; Rainer, W.—“Chitosan: A New Hemostatic,” Annals of Thoracic Surgery, vol. 36, No. 1, 1983, 55-58.
McDowell, R. H.—Properties of Alginates, Alginate Industries Limited; Second Edition, 1961 Bedford Street, Strand, London England.
McRight, A. E. (1998) 510(k) Summary of Safety and Effectiveness (unpublished report submitted to the FDA), 2 pp.
The Merck Index, 7^{th} Edition. Stecher, P. G., Ed. Rahway, N.J. Merck & Co., Inc., 1960
Mi, F L; Wu, Y B; shyu, S S; Schoung, J Y; Huang, Y B; Tsai, Y H; Hao, J Y—“Control of wound infections using a bilayer chitosan wound dressing with sustainable antibiotic delivery,” J Biomed Mater Res. 2002 Mar. 5; 59(3): 438-49.
Muzzarelli, R.; Baldassarre, V.; Conti, F.; Ferrara, P.; Biagini, G.—“Biological Activity of Chitosan: Ultrastructural Study,” Biomaterials, vol. 9, May 1988, 247-252.
Muzzarelli, R.; Tarsi, R.; Filippini, O.; Biagini, G.; Varaldo, P.—“Antimicrobial Properties of N-Carboxybutyl Chitosan,” Antimicrobial Agents and Chemotherapy, vol. 34, No. 10, October 1990, 2019-2023.
Pusateri, Anthony E. McCarthy, Simon J.; Gregory, Kenton W.; Harris, Richard A.; Cardenas, Luis; McManus, Albert T.; Goodwin, Cleon W. Jr.—“Effect of a chitosan-Based Hemostatic Dressing on Blood Loss and Survival in a Model of Severe Venous Hemorrhage and Hepatic Injury in Swine,” Journal of Trauma-Injury Infection & Critical Care, 54(1): 177-182, January 2003.
Rao, S. B. and Sharma, C. P.—“Use of chitosan as a biomaterial: studies on its safety and hemostatic potential” J Biomed Mater Res. 1997 January; 34)1): 21-8.
Silvetti, A. N.—An Effective Method of Treating Long-Enduring Wounds and Ulcers by Topical Application of Nutrients.” J. Dermatol, Surg. Oncol. 1981-7:501-508.
Silvetti, A. N.—“Mechanisms Involved in Wound Healing”—Faseb Journal 1993 (3-A5956) A1251.
Silvetti, A. N.—Polysaccharides as Effective Chemo-attractants to White Blood Cells and Macrophages. Federation proc. 1987 (46 A3868) 980.
Spectrum Laboratory Products, Inc. Gardena, Calif.—MSDS Sheet, Sep. 13, 2006.
Supfle, K. and Werner, R. (1951 Microdeosimetric investigation of the olygodynamic effect of silver. Mikrochemie ver. Mikrochim. Acta., 36/37, 866-881.
Taber's Cyclopedic Medical Dictionary, 18^{th} Edition, F. A. Davis Company, Philadelphia, Pa.
Tainan Science Industrial Park, No. 10, Lane 31, Sec. 1, Huandong Road, Tainan County, Taiwan.
The United States Pharmacopeia of America—Eighteenth Revision, Sep. 1, 1970, p. 02-103.
Trop, Marija, et al. Silver-Coated Dressing Acticoat Caused Raised Liver Enzymes and Argyria-like Symptoms in Burn Patient. The Journal of TRAUMA Injury, Infection, and Critical Care—Volume 60-Number 3:648-652, March 2006.
Wang, Xiaohong; Yan, Yongnian; Zhang, Renji—“A Comparison of Chitosan and Collagen Sponges as Hemostatic Dressings” Journal of Bioactive and Compatible Polymers, Vol. 21, No. 1, 39-54, 2006.
Wolberg A S (2007). “Thrombin generation and fibrin clot structure”. Blood Rev 21 (3) 131-42. doi:10.1016/j.blre.2006.11.001
Z-Medica Corporation—QuikClot 510(k) K070010
Z-Medica Corporation—QuikClot 510(k) K061767
Z-Medica Corporation—QuikClot 510(k) K021581

U.S. PATENTS

| | | |
|---|---|---|
| 4,394,373 | Jul. 19, 1983 | Malette et al. |
| 6,967,261 | Nov. 22, 2005 | Soerens et al. |
| 5,674,524 | Oct. 7, 1997 | Scherr |
| 5,718,916 | Feb. 17, 1998 | Scherr |
| 7,128,929 | Oct. 31, 2006 | Scherr |
| 6,696,077 | Feb. 24, 2004 | Scherr |
| 5,147,648 | Sep. 15, 1992 | Bannert |
| 5,688,923 | Nov. 18, 1997 | Gerrish |
| 3,639,575 | Feb. 1, 1972 | Schmolka |
| 2,674,619 | Apr. 6, 1954 | Lundsted |
| 4,184,974 | Jan. 22, 1980 | Van Leuven |

FOREIGN PATENTS

| | | |
|---|---|---|
| 221859 (India) | Jul. 8, 2008 | Scherr |
| 2,357,765 (Great Britain) | Apr. 21, 2004 | Scherr |
| 2322266 (Russia) | Feb. 18, 2003 | Scherr |

I claim:

1. The process for making a cation cross-linked pectin composition comprising:
a) Cross-linking the pectin with an aqueous soluble silver salt in distilled or deionized water and,
b) with continuous stirring, adding to the silver pectate suspension, a plasticizing agent and,
c) a surface active agent and,
d) a hydrophilic polymer and,
e) an aqueous soluble calcium salt.

2. The process of claim 1 wherein a non-pectin polysaccharide is added to the silver pectate composition.

3. The process of claim 2 wherein the non-pectin polysaccharide is selected from the group consisting of carboxymethylcellulose, carboxymethyl ethyl cellulose, hyaluronic acid, carrageenan, and/or gellan gum.

4. The process of claim 3 wherein the non-pectin polysaccharide is carboxymethylcellulose.

5. The process of claim 1 where the aqueous soluble silver salt may be silver acetate, silver fluogallate, silver nitrate, and/or silver sulfate.

6. The process of claim 1 wherein the aqueous soluble calcium salt added to the silver pectate composition may be calcium gluconate, calcium chloride, calcium chlorate, calcium lactate, calcium nitrate, calcium propionate, and/or calcium thiosulfate.

7. The process of claim 6 wherein the aqueous soluble calcium salt is calcium gluconate.

8. The process of claim 1 wherein the upper limit of the DE of the pectin that is cross-linked with the silver is 50 percent.

9. The process of claim 1 wherein the upper limit of the DE of the pectin that is cross-linked with the silver is 30 percent.

10. The process of claim 1 wherein the upper limit of the DE of the pectin that is cross-linked with the silver is 5 percent.

11. The process of claim 1 wherein the lower limit of the DE of the pectin that is cross-linked with the silver is 0 percent.

12. The process of claim 1 wherein the upper limit of the DA of the calcium sensitive pectin is less than 50 percent.

13. The process of claim 1 wherein the upper limit of the DA of the calcium sensitive pectin is less than 30 percent.

14. The process of claim 1 wherein the upper limit of the DA of the calcium sensitive pectin is less than 15 percent.

15. The process for making a cross-linked silver pectin powder composition comprising:
a) Drying the composition of claim 1 and,
b) grinding the dried contents in a mill and,
c) processing the dried contents through a mesh screen.

16. The process of claim 15 where in the cross-linked pectin composition is dried in an oven at 85-90° C.

17. The process of claim 16 wherein the dried contents are ground in a mill to a fine powder.

18. The process of claim 17 wherein the dried powder is sieved through a 100 mesh screen.

19. The process of making a cross-linked silver pectate dressing comprising:
a) adding the silver pectate powder composition prepared in accord with claims 15-17, to a quantity of deionized or distilled water and,
b) adding glycerin and,
c) adding a surface active agent and,
d) adding an aqueous soluble calcium salt and,
e) adding maltodextrin and,
f) adding a hydrophilic polymer and,
g) adding a plasticizing agent.

20. The process of claim 19 wherein the plasticizing agent is selected from the group consisting of carboxymethylcellulose, carboxymethyl ethyl cellulose, hyaluronic acid, carrageenan, and/or gellan gum.

21. The process of claim 20 wherein the non-pectin polysaccharide is carboxymethylcellulose.

22. The process of claim 21 wherein the carboxymethylcellulose is of low viscosity.

23. The process of claim 19 wherein the aqueous soluble calcium salt added to the silver pectate composition may be calcium gluconate, calcium chloride, calcium chlorate, calcium lactate, calcium nitrate, calcium propionate, and/or calcium thiosulfate.

24. The process of claim 23 wherein the aqueous soluble calcium salt is calcium gluconate.

25. The process of claim 19 wherein the surface active agents may be selected from a group of Tweens: Tween 20, polyoxyethylene sorbitan monolaurate; Tween 40, polyoxyethylene sorbitan monopalmitate; or Tween 85, polyoxyethylene sorbitan trioleate; and or sodium lauryl sulfate.

26. The process of claim 25, wherein the surface active agent is Tween 80.

27. The process of claim 25 wherein the surface active agent is sodium lauryl sulfate.

28. The process of claim 19 wherein the hydrophilic polymer is sodium polyacrylate.

29. The process of any preceding claim wherein the said silver pectate foamed composition may be layered onto a surface that will permit the evaporation of water so resulting in a sheet of foamed silver pectate composition.

30. The process of claim 29 wherein the surface on to which the silver pectate composition is poured is a fibrous cloth.

31. The process of claim 29 wherein said fibrous cloth is selected from cloths prepared from cotton, polyester, wool, nylon, rayon, or mixtures thereof.

32. A silver pectate wound dressing suitable for direct application to a wound comprising a backing having layered thereon a foamed silver pectate composition produced by any of the previous claims.

33. A silver pectate hemostatic powder suitable for direct application to a wound produced by any of the previous claims.

34. The process of any preceding claim wherein a medicament is added to the silver pectate composition.

35. The process of claim 34 wherein said medicament is selected from the group consisting of collagen, maltodextrin, antibiotics, antibacterial agents, anti-inflammatory agents, ascorbic aid, amino acids, and/or mixtures thereof.

36. A silver pectate moiety.
